(19) United States
(12) Patent Application Publication
(10) Pub. No.: US 2009/0062833 A1
(43) Pub. Date: Mar. 5, 2009

(54) Device and method for placement of interbody device

(75) Inventor: John K. Song, Chicago, IL (US)

Correspondence Address: JOHN K. SONG, 474 NORTH LAKE SHORE DR., APT. # 4508, CHICAGO, IL, 60611-6465, US

(73) Assignee: Vermillion Technologies, LLC, Chicago, IL (US)

(21) Appl. No.: 11/895,651

(22) Filed: Aug. 27, 2007

(51) Int. Cl. A61B 17/00; A61B 17/58

(52) U.S. Cl. 606/191; 606/103; 606/90

(57) ABSTRACT

A method for performing percutaneous interbody preparation and placement of an interbody device is disclosed. The method includes the steps of inserting dilators into the interbody space and then working through a tube to prepare the interbody space and place an implant. Once the device is placed, the tube is removed.

### THE INVENTION

The present invention relates to a device for percutaneously preparing an interbody space and placing a device within the interbody space of the spine.

### BACKGROUND OF THE INVENTION

Spinal fusion surgery is the joining together of vertebrae of the spine. The underlying concept of fusion surgery implants is to maintain the relative position of the vertebral bodies with respect to eachother while bone graft placed between the vertebrae has an opportunity to heal and mature. These devices employ different strategies and philosophies, such as devices which utilize the pedicles, and devices which are placed in the disc space. The latter devices and techniques associated with these devices are known as “interbody fusion” devices. While no single technique has been universally accepted as the most optimum method, there is growing evidence that interbody fusion may be the preferred method.

Initially, all interbody fusion procedures were accomplished using a posterior approach. The procedure typically begins with a laminectomy, followed by removal of the intervertebral disc, and then in most cases the height of the disc space is increased and packed with pieces of bone. The goal is for the inserted bone pieces to fuse together with the vertebrae above and below that disc space, forming a bridge of bone and therefore eliminating motion at that spinal level.

More recently, interbody fusion performed by an anterior procedure has gained popularity. Anterior fusion instruments typically provide for a retroperitoneal or transperitoneal approach to the lumbar disc. Some or all of the disc is removed, and either bone or a metallic device is placed into the disc space. These devices also typically provide a means for distracting the disc space, i.e. making the space between the discs wider. Presently, this aspect of lumbar interbody fusion procedures are considered to be an important step in the procedure because of its effects on the neural foramina, or areas from which the nerve roots exit through the vertebra. It is generally accepted that enlarging the disc space consequently enlarges the neural foramina, thus decompressing the exiting nerve roots.

Due to presently available equipment, current anterior interbody fusion surgeries are disadvantageously invasive, requiring large incisions and manipulation of both tissue and organs. While attempts have been made to perform anterior interbody fusions laparoscopically, these procedures are often complicated and are typically performed under general anesthesia.

Therefore, a need exists for an interbody fusion method which reduces trauma to the patient; consequently reducing recovery time. Implant and instruments for percutaneous anterior interbody fusion; enabling the surgeon to distract the disc space to restore disc height, maintain the distraction, and promote the growth of interbody bone graft, would satisfy this heretofore unaddressed need.

Most commonly, surgery over a wire or through a tube has been used for placement of devices into bones of the spine. However, the technique of the current invention has not been applied for disc space preparation and device placement.

Numerous prior art methods and devices exist in this arena. Some describe utilization of wires or tubes for percutaneous placement of interbody devices—however, most do not employ direct entry into the interbody space itself with percutaneous instruments. U.S. Pat. No. 6,666,891 to Boehm et al describes a system whereby a guide wire is placed into the discspace and a sequence of serial dilators is used to create a working channel through which disc space preparation is performed. An expandable interbody fusion device is then placed and the guide tube removed.

The current invention describes a method and means of placing an interbody device percutaneously into the space between vertebrae with a substantially posterior, posterolateral, or lateral approach. The device itself may comprise a device for purposes of fusion or for other purposes such as nucleus or disc replacement or placement of biologic substances. One embodiment of the current invention differs significantly from that of Boehm et al in that the need for a guide wire and an expandable interbody device are eliminated. The working tube of the present invention allows preparation of the endplates of the vertebra through windows in the side. This allows more stable placement of a longer tube into the disc space while maintaining access to the endplates. In yet another embodiment, the working tube with side openings also becomes the delivery device and implant. The present invention includes methods and devices for both fusion and non-fusion spinal surgeries.

U.S. Patent Documents

| | | |
|---|---|---|
| 3811449 | May 1974 | Gravlee et al. |
| 5015247 | May 1991 | Michelson |
| 5522899 | June 1996 | Michelson |
| 5609635 | March 1997 | Michelson |
| 5665122 | September 1997 | Kambin |
| 5782832 | July 1998 | Larsen et al. |
| 6083225 | July 2000 | Winslow et al. |
| 6113602 | September 2000 | Sand |
| 6126689 | October 2000 | Brett |
| 6129763 | October 2000 | Chauvin et al. |
| 6395034 | May 2002 | Suddaby |
| 6419705 | July 2002 | Erickson |
| 6527734 | March 2003 | Cragg et al. |
| 6666891 | December 2003 | Boehm et al. |

Foreign Patent Documents

| | | |
|---|---|---|
| 44 16 605 | November, 1994 | DE |
| 98 10832 | August, 1998 | FR |
| WO 96/27321 | September, 1996 | WO |
| WO 00/35388 | June, 2000 | WO |
| WO 00/49977 | August, 2000 | WO |

### SUMMARY OF THE INVENTION

While the invention has been shown and described with reference to certain preferred embodiments, it will be understood by those skilled in the art that various changes and modifications in form and detail may be made therein without departing from the spirit and scope of the invention, as defined by the appended claims.

It is an object of the present invention to provide a percutaneous method and device for interbody spinal procedures which facilitates placement of said device for the surgeon, reduces trauma to the patient, and reduces recovery time.

It is also an object of the present invention to provide a method of placing a percutaneous interbody device through small incisions in the body of the patient and utilizes a minimum of incisions to complete the procedure.

It is an object of the present invention to present methods and devices for percutaneously preparing an interbody space for an implant. Such an implant may be used for fusion or non-fusion procedures or to deliver drugs or other biologic compounds.

It is a further object of the present invention to provide a device which facilitates the percutaneous interbody fusion procedure.

It is yet another object of the present invention to provide a device which distracts the disc space and which may be inserted over a guide wire.

It is yet another object of the invention to provide a typical example of a method and instruments for preparation of the interbody space for receiving said interbody device.

A wide variety of instruments and the sequence of their use can be constructed. For the purposes of example only the following is given for reference:
1) A guide wire, if desired, can be placed through a trocar or by other means familiar to the practitioner. Placement may be facilitated by fluoroscopy or other navigation techniques.
2) An initial dilator, which is tapered at the leading edge to facilitate entry into the disc space and to push delicate nerves aside, is placed in the disc space (over the guide wire if used). Additional concentric dilators with increasing diameters (serial dilators) may be used to increase the size of the working corridor. The diameter of the appropriately sized final dilator is noted and all dilators except the initial dilator are removed. If a guide wire is used, it may be removed now or after step 3.
3) The appropriately sized working tube is selected based on the final dilator used in step 2 and is placed over the initial dilator and introduced into the disc space such that the openings in the working tube face the endplates.
4) The initial dilator/guide wire is removed.
5) Instruments can then be used from within the working tube to remove disc material and prepare the endplates if necessary.
6) Depending on the embodiment, a fixed-size implant can be placed through the working tube and into the disc space. The implant can be filled with bone graft if necessary either before or after implantation. The working tube is removed once the implant is in place.
7) In another embodiment, the final implant forms the leading end of the working tube. Once the interbody preparation is complete, the device can be filled with bone graft (if needed) and the rest of the working tube detached and removed.

A used above, the term concentric dilators may refer to any tubular structure of any cross sectional profile. Additionally, partially formed tubes, parts of tubes, or tubes with slits or cutouts in the side wall may be used.

A kit for performing percutaneous interbody preparation is also provided, which includes the guide wire and means for placing the guide wire, the instruments required to perform the disc space preparation and disc removal, working tubes, and possibly also the device itself and its application handle.

In this specification, the terms “disc space” and “interbody space” are intended to be used interchangeably despite technical differences. Overall, the terms are substantially meant to include the space between two vertebra.

Lastly, all instruments and devices could potentially incorporate means of surgical tracking and intra-operative nerve monitoring to minimize injury to the nervous structures.

### BRIEF DESCRIPTION OF THE DRAWINGS

FIG. 1A shows an isometric view of two vertebra with disc space and the position of initial guide wire.

FIG. 1B shows a lateral view of the guide wire in place in the disc space. Only one vertebra is shown for simplicity.

FIG. 1C shows an axial view of the guide wire in the disc space and the trajectory the guide wire takes to enter the disc space.

FIG. 2A shows an isometric view of the initial dilator entering the disc space over the guide wire.

FIG. 2B shows a lateral view of the same.

FIG. 2C shows an axial view of the initial dilator in the disc space.

FIG. 3A shows an isometric view of the working tube over the initial dilator in the disc space.

FIG. 3B shows a lateral view of the same.

FIG. 3C shows an axial view of the working tube in the disc space.

FIG. 4A shows an isometric view of the working tube in the interbody space with the guide wire and initial dilator removed.

FIG. 4B shows a lateral view of the working tube in the interbody space.

FIG. 4C shows an axial view of the working tube in the interbody space.

FIG. 5A shows an isometric view of an example instrument used through the tube.

FIG. 5B shows an expanded view of the tip of the working tube in the interbody space and the example instrument.

FIG. 5C shows a lateral view of the same.

FIG. 5D shows an axial view of the working tube and example instrument in place.

FIG. 6A shows an isometric view of the interbody space with the final implant.

FIG. 6B shows an axial view of the final implant in the interbody space.

### DETAILED DESCRIPTION OF THE DRAWINGS

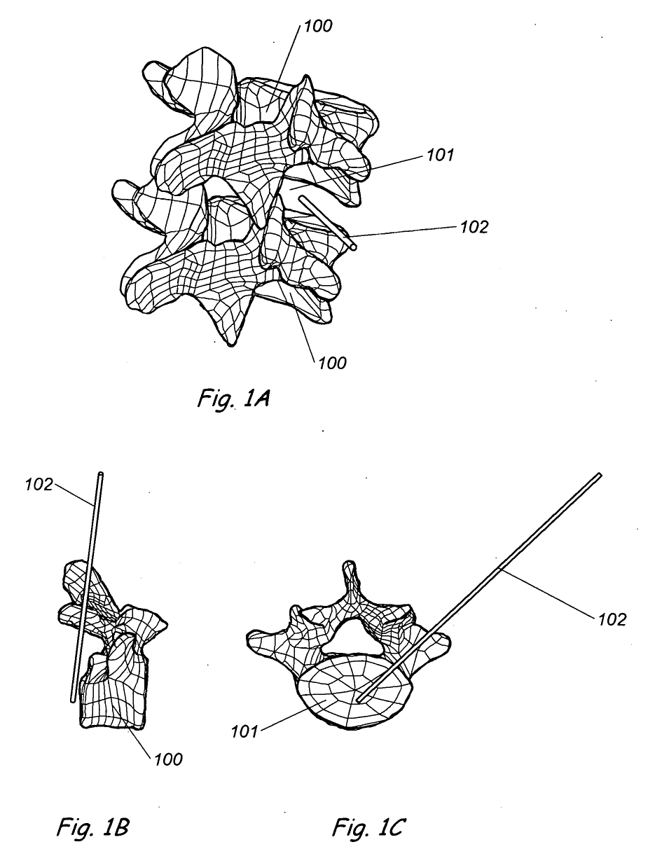

FIGS. 1A-1C shows the position of the initial guide wire (102) positioned in the disc space (101) between two vertebrae (100). Introduction of the guide wire is accomplished thorough a larger bore needle such as a “Jamshidi” needle or other trocar. Note that placement of the guide wire is outside of the facet complex. In one embodiment, no initial guide wire is used and the surgical method begins with placement of the initial dilator.

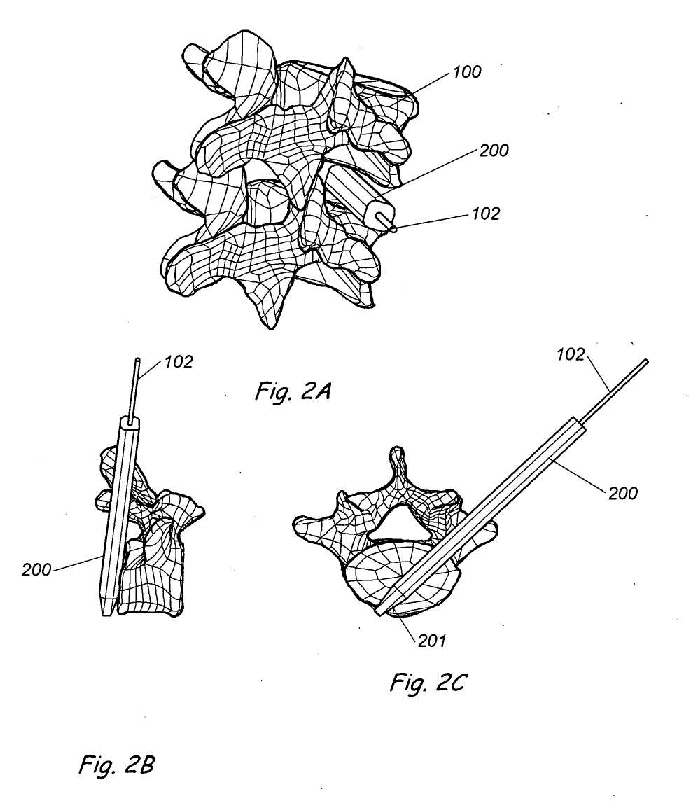

FIGS. 2A-2C shows an initial dilator (200) which, in this case, is placed over the guide wire (102) if used. No limitation on whether or not a guide wire is used is intended by this representation. The initial dilator has a tapered tip (201) to ease passage into the disc space and other soft tissue.

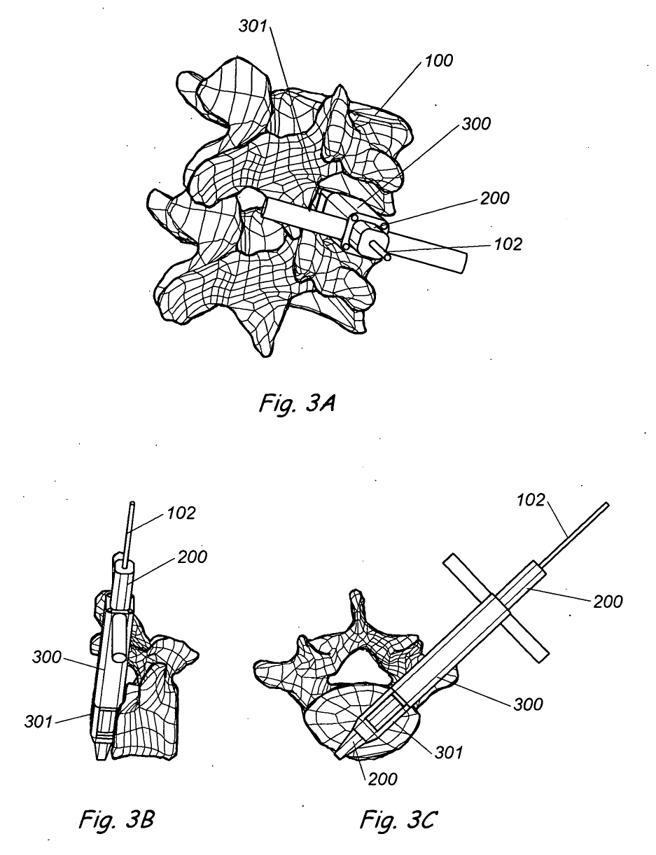

FIGS. 3A-3C shows a working tube (300) placed over the initial dilator into the interbody space (101). Note that the leading end (301) (the end in the interbody space) is constructed with openings on the end and sides. In one embodiment, leading end 301 is an integral part of the working tube and in yet another embodiment the leading end 301 is detachable from the working tube and becomes the final implant.

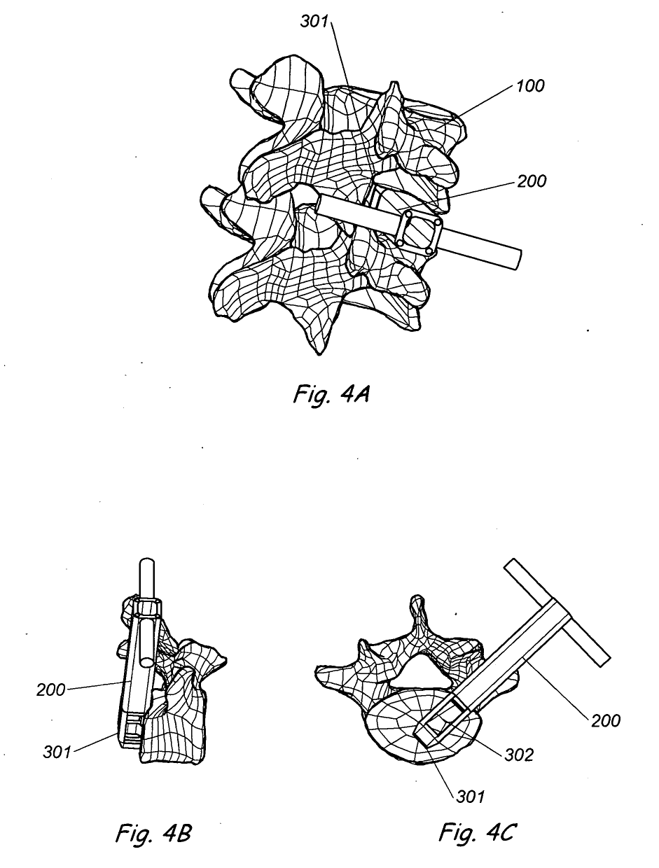

FIGS. 4A-4C shows the working tube (300) and leading end (301) in the interbody space with the initial dilator and guide wire removed. Note that leading end (301) has openings in the sides (302) which allow access to the surrounding disc material and endplates of the vertebra.

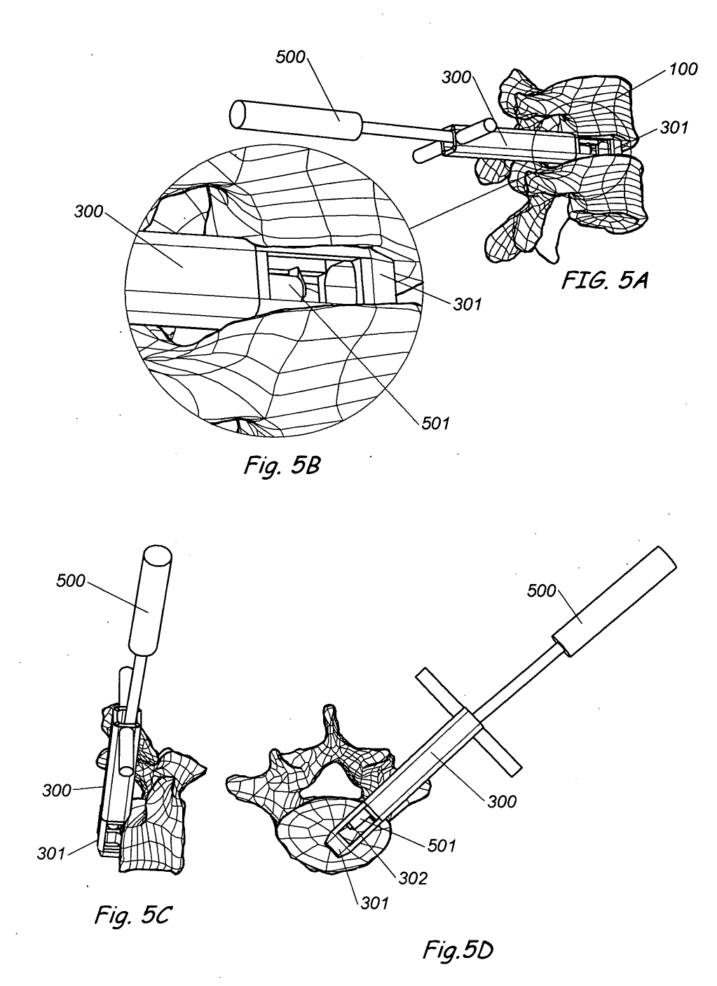

FIGS. 5A-5D show the working tube (300) and leading end (301) between two vertebrae (100). An example instrument (500) that can be used down the working tube is shown (500). FIG. 5B shows a detail view of the leading end (301) depicting how the tip (501) of the example instrument (500) can be used to prepare the endplate through opening 302. Various instruments known to the practitioner can be used including but not limited to curettes, rongeurs, graspers, drills, forceps, rasps, chisels, etc. These instruments can be used for preparing endplates, such as removing disc material.

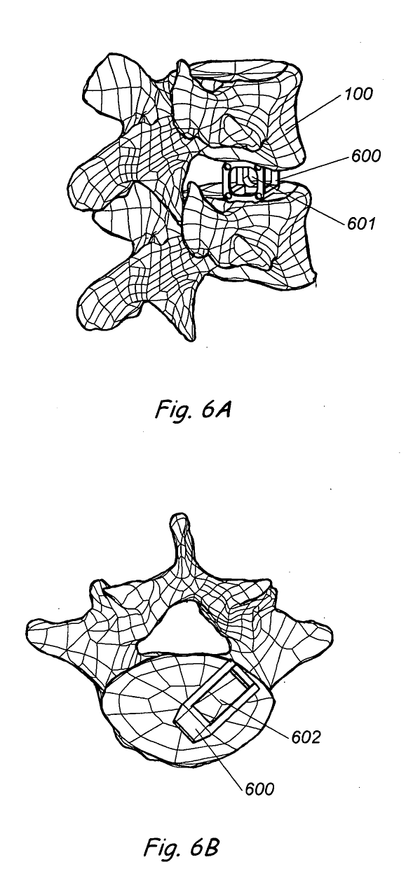

FIGS. 6A and 6B shows what the final implant (600) may look like. Depicted are the central cavity for bone graft or other biologic material (601) and side openings (602) to allow the material within the implant to interact with the surrounding interbody space. In one embodiment, the final implant (600) is a separate device which is placed through the working tube. In this embodiment, the final device is small enough to fit down the center of the working tube and out the leading end. In another embodiment, the final implant (600) is actually part of the working tube and is simply detached from the rest of the working tube once finished (in which case it would be synonymous with feature 301 from FIGS. 3,4, 5).

The entire procedure may be repeated on the contralateral side.

What is claimed is:

1. A method for dilating the space between two bones or parts of a single bone by means of two or more concentric and mating dilators placed in a sequentially increasing manner between said bones to create a working space.

2. Method of claim 1 where a wire is used to guide at least one of said dilators.

3. Method of claim 1 where said mating dilators are concentric tubes

4. Method of claim 1 where said mating dilators are partial tubes which are concentric

5. Method of claim 1 where said mating dilators are formed to facilitate passage through soft tissue.

6. Method of claim 1 where said mating dilators are of any cross-sectional profile

7. A method and devices for preparation of an interbody space to accept an implant comprising the following steps:
Distraction of a space between two bones by placement of one or more dilators into the interbody space; Placement of a hollow working tube over the dilator(s) into the interbody space; Removal of the dilators; Preparation of the interbody space through one or more openings in the working tube; Placement of an implant through the working tube into the interbody space; Removal of the working tube.

8. Method of claim 7 where a wire is used to guide the initial dilator into position.

9. Method of claim 7 where the instruments used to prepare the interbody space are commonly used surgical instruments.

10. Method of claim 7 where the dilator(s) can be used to determine an appropriately sized working tube.

11. Method of claim 7 where said side openings of the working tube may be used to remove disc material.

12. Method of claim 7 where said openings in the working tube may be used to prepare vertebral endplates.

13. Method of claim 7 where the procedure may be performed unilaterally or bilaterally.

14. Method of claim 7 where the hollow working tube is substantially cylindrical.

15. Method of claim 7 where the hollow working tube is substantially non-cylindrical.

16. Method of claim 7 where the implant is a fusion device.

17. Method of claim 7 where the implant is a non-fusion device.

18. A method and device for preparing an interbody space to accept an implant comprising the following steps:
Distraction of the space between two bones by placement of one or more dilators into the interbody space; Placement of a hollow working tube attached to an implant over the dilator(s) into the interbody space; removal of the dilators; Preparation of the interbody space through one or more openings in the implant; Detachment of the working tube from the implant; Removal of the working tube.

19. Method of claim 18 where a wire is used guide the initial dilator into position.

20. Method of claim 18 where the instruments used to prepare the interbody space are commonly used surgical instruments.

21. Method of claim 18 where the dilator(s) can be used to determine an appropriately sized working tube.

22. Method of claim 18 where said openings of the implant may be used to remove disc material.

23. Method of claim 18 where said openings of the implant may be used to prepare vertebral endplates.

24. Method of claim 18 where the procedure may be performed unilaterally or bilaterally.

25. Method of claim 18 where the hollow working tube is substantially cylindrical.

26. Method of claim 18 where the hollow working tube is substantially non-cylindrical.

27. Method of claim 18 where the implant is a fusion device.

28. Method of claim 18 where the implant is a non-fusion device.

29. A method and device for preparing an interbody space to accept an implant comprising the following steps:
Distraction of the space between two bones by placement of one or more dilators into the interbody space; Placement of a hollow working tube over the dilator(s) into the interbody space; Removal of the dilators; Preparation of the interbody space; Placement of a non-expandable fusion implant through the working tube into the interbody space; Removal of the working tube.

30. Method of claim 29 where a wire is used to guide the initial dilator into position.

31. Method of claim 29 where the instruments used to prepare the interbody space are commonly used surgical instruments.

32. Method of claim 29 where the dilator(s) can be used to determine an appropriately sized working tube.

33. Method of claim 29 where the procedure may be performed unilaterally or bilaterally.

34. A method and device for preparing an interbody space to accept an implant comprising the following steps:
Distraction of the space between two bones by placement of one or more dilators into the interbody space; Placement of a hollow working tube over the dilator(s) into the interbody space; Removal of the dilators; Preparation of the interbody space; Placement of a non-fusion implant through the working tube into the interbody space; Removal of the working tube.

35. Method of claim 34 where a wire is used to guide the initial dilator into position.

36. Method of claim 34 where the instruments used to prepare the interbody space are commonly used surgical instruments.

37. Method of claim 34 where the dilator(s) can be used to determine an appropriately sized working tube

38. Method of claim 34 where the non-fusion implant is expandable.

39. Method of claim 34 where the non-fusion implant is non-expandable.
